(19) United States
(12) Patent Application Publication
(10) Pub. No.: US 2012/0166214 A1
(43) Pub. Date: Jun. 28, 2012

(54) METHOD AND SYSTEM FOR MANAGING WORK LISTS FOR MEDICAL IMAGING PROCEDURES

(76) Inventors: Naveen Benagi, Dharwad (IN); Raja Shekhara, Bangalore (IN)

(21) Appl. No.: 13/327,853

(22) Filed: Dec. 16, 2011

(30) Foreign Application Priority Data
Dec. 27, 2010 (IN) 3963/CHE/2010

(51) Int. Cl. G06Q 50/22; G06Q 10/06

(52) U.S. Cl. 705/2

(57) ABSTRACT

A work list management method and system is disclosed herewith. The method of maintaining a work list for medical imaging procedures comprises accessing at least one data source by a processor for job information relating to medical imaging procedures; automatically refreshing a work list in regular intervals; the work list being displayed to a clinician performing the imaging procedure; and generating at least one alert based on the new job information in the work list.

### BACKGROUND OF THE INVENTION

1. Field of the Invention

This disclosure relates generally to managing work lists and, more particularly to, method and system for auto populating and maintaining work list for medical imaging procedures without human intervention.

2. Description of the Prior Art

Conducting medical imaging procedures in an efficient manner in large hospitals are becoming a challenge. The clinicians who are performing the imaging procedures have to refresh a work list that indicates scheduled medical procedures periodically to update the work list. In today's scenario, one common tedious job done by the clinician is to repetitively refresh the work list to populate the new job information into the work list. Usually the receptionist in the hospital, records the patient information as soon as patient walks into the hospital and if required schedules the patient to different clinicians for imaging procedures. Eventually this patient information will be integrated into the any data repository.

When a clinician logs in a system, a work list is displayed to the clinician and the clinician manually refreshes the work list and gets information about all the patients that are scheduled for the procedure on the work list. When the clinician completes all the assigned imaging procedures in work list, he again refreshes the work list to know next set of new scheduled patients. Until the clinician refreshes the work list, the work list will not be populated and the clinician will not have any information about the scheduled patients.

Many times the clinician may forget to refresh the work list and may miss on completing the scheduled procedures. Further, until the clinician refreshes the work list, he is unaware of the number of patients that needs to be attended in a scheduled time and hence the clinicians may not be able to plan the procedures accordingly.

It will be beneficial to alert a clinician about the pending patients and the priority or any other special attention that has to be provided to a patient while imaging. This will help the clinician to perform the imaging procedures more efficiently. However currently, until the clinician refreshes the work list, he will not have access to information about the scheduled procedures and the work list does not populate any additional patient information relating to his priority or preferences. Thus the clinician is not able to prioritize the patient, if required.

Further, in large hospitals there will be different clinicians performing imaging procedures and currently there are no effective ways to identify additional information about a job information in the work list or on scheduled patient. For example, if there is a change in the clinician who is performing the procedure, the new clinician should be able to get an idea about the pending patients, actions taken by the earlier clinician such as keeping some patients on hold, rejecting some job information etc. Thus it will be beneficial to have an alert indicating these, so that the clinician who is performing the procedure will be able to interpret the work list and the job information provided in the work list in a better way.

Thus, a need exists for a method and system for managing work lists for a medical imaging procedure.

### BRIEF SUMMARY OF THE INVENTION

The above-mentioned shortcomings, disadvantages and problems are addressed herein which will be understood by reading and understanding the following specification.

One embodiment of the present invention provides a method of maintaining a work list for medical imaging procedures. The method comprises: accessing at least one data source by a processor for job information relating to medical imaging procedures; automatically refreshing a work list in regular intervals; the work list being displayed to a clinician performing the imaging procedure; and generating at least one alert based on the new job information in the work list.

In another embodiment, a method of managing a mammography work list is disclosed. The method comprises: accessing data sources for schedule information relating to mammography exam; automatically refreshing a work list in a regular interval; the work list being displayed to a clinician performing the exam; displaying the schedule information along with patient information for a predefined period; and representing the schedule information using an image icon after expiring the predefined period.

In yet another embodiment, a work list management system for medical imaging operation is described. The system comprises: an interface for a clinician to login and access work list; a processor having an access module configured to access at least one job storage device; an update module for updating the work list in substantially real time; visual module configured to alert the clinician about an new job information in the work list; and a display for displaying the work list.

Various other features, objects, and advantages of the invention will be made apparent to those skilled in the art from the accompanying drawings and detailed description thereof.

### BRIEF DESCRIPTION OF THE DRAWINGS

FIG. 1 is a flowchart illustrating method of maintaining a work list for medical imaging procedures as described in various embodiments of the invention;

FIG. 2 is a flowchart illustrating detailed workflow for handling an imaging procedure as described in various embodiments of the invention;

FIG. 3 illustrates a work list displayed with image icons as described in various embodiments of the invention;

FIG. 4 is a flowchart illustrating method of managing mammography work list as described in an embodiment of the invention; and

FIG. 5 shows a work list management system as described in an embodiment of the invention.

### DETAILED DESCRIPTION OF THE INVENTION

In the following detailed description, reference is made to the accompanying drawings that form a part hereof, and in which is shown by way of illustration specific embodiments that may be practiced. These embodiments are described in sufficient detail to enable those skilled in the art to practice the embodiments, and it is to be understood that other embodiments may be utilized and that logical, mechanical, electrical and other changes may be made without departing from the scope of the embodiments. The following detailed description is, therefore, not to be taken as limiting the scope of the invention. To the extent that the figures illustrate diagrams of the functional blocks of various embodiments, the functional blocks are not necessarily indicative of the division between hardware circuitry. Thus, for example, one or more of the functional blocks may be implemented in as single unit. It should be understood that the various embodiments are not limited to the arrangements and instrumentality shown in the drawings.

In various embodiments, work list management methods and systems are proposed. The work list is auto populated at regular intervals and the clinician is alerted about a new job information in the work list. The new job information includes details of the patients scheduled for an imaging procedure for a scheduled time.

Though the method and system are explained with reference to medical imaging work lists, the method and system can be extended to any work list and this will help a user to auto populate the workflow and alert the user about the new items in the work list.

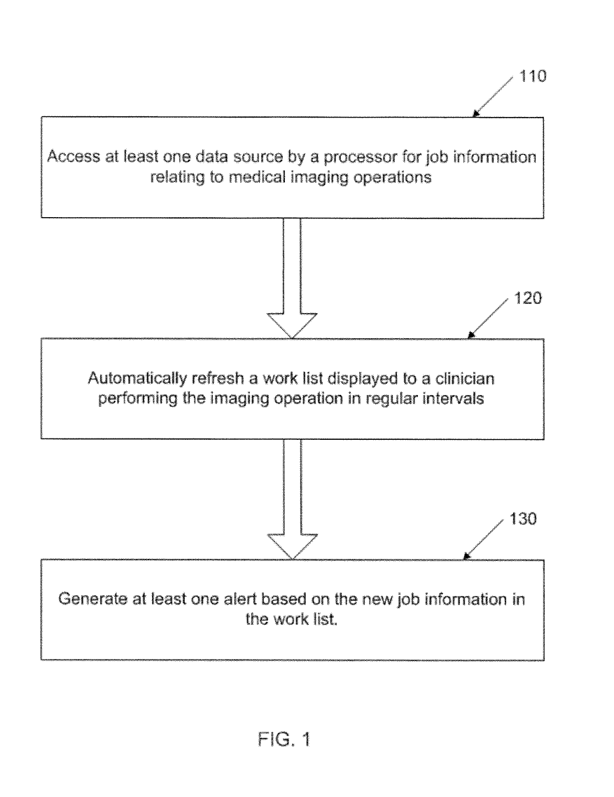

FIG. 1 is a flowchart illustrating method of maintaining a work list for medical imaging procedures as described in various embodiments of the invention. At step 310 data sources are accessed by a processor to obtain job information relating a medical imaging procedure. The data sources could include any data repository where job information such as details of patients requires imaging procedures is stored. Generally a receptionist at the hospital obtains patient information and imaging procedures are scheduled and uploaded in a Hospital Information System (HIS) or RIS. The processor, queries the data sources automatically on a regular interval for any new job information. In a hospital, there could be many clinicians performing the imaging operations and data sources will have job information corresponding to each clinician. At step 120, a work list is automatically refreshed and populated with new job information. The work list is displayed to a clinician who is performing the imaging operation and the work list is refreshed automatically. At step 130, the clinician is alerted about the new job information populated into the work list. The clinician can be alerted by visual alerts or by audio alerts.

In an embodiment, the alert is visual alert displayed on a display to the clinician where the work list is being displayed. While populating the job information in the work list, the patient information along with certain other details could be displayed on a screen where the work list is displayed. This patient information may be displayed for a predetermined period and then could be represented as an image icon. The icon will represent new job information in the work list.

In an embodiment, along with the image icon, a number field could be provided to indicate the number of new job information in the work list or number of patients scheduled could be displayed.

In an embodiment, additional alerts could be generated based on nature of the patient information. The audio alert or the icon representing the visual alert can be selected based on patient information. Different types of alerts may be generated based on the clinical relevance of the patient information. This alert could be displayed along with the icon representing the new job information. For example, if a patient needs to be scanned urgently, the job information may be populated to the work list and may be displayed as an icon representing a priority job information which indicates that the job information is urgent and treated with high priority. In another example, patient information could include prior clinical history of the patient about which the clinician has to be aware while performing the imaging operation. In an example, if the patient has any implants, while populating the job information, an alert indicating the same could be generated. In the event of visual alerts, different image icons could be used to represent the alert. For example, along with icon, there could be a text area which could represent nature of the alert or different icons could be used to represent different alerts. Further different types of icons could be used to indicate age group, gender, existing or new patients or any other patient information. Thus based on the patient information including clinical and non clinical information, additional alerts may be generated and represented using different types of additional icons or audio alerts.

In an embodiment, an alert could be maintained until the clinician attends the job information. The alert could be maintained until the operator completes or accesses the job information. Thus the new job information icon will be removed only when the clinician completes all the jobs listed in the work list.

In an embodiment, the additional icons generated based on the patient information or based on the actions taken on the new job information can be modified. For example, the icon can be modified based on the actions taken by the clinician on the job information. For example, if the imaging procedure is in progress, it could be presented by a different icon, than from an icon which represents un attended job information in the work list. The icon changes automatically upon taking action on the job information and no icon will be displayed if all the job information in the work list is accessed and completed. If new job information with high priority icon is completed, the priority icon can be removed from the display.

In an embodiment, an image icon will represent the new job information and this will be displayed until the operator accesses all new job information in the work list. Alternately, the image icon could be displayed until the clinician completes the action i.e. imaging procedure on the job information.

In an embodiment, the clinician may transfer some job information to the data source or to a receptionist who will be able to reschedule the imaging procedures. In this event, the job information transferred will be deleted from the current work list automatically. In case of over load situation, the clinician can reject a job information and upon rejecting the same, it will be directed to HIS/RIS or to the receptionist and based on other clinician's availability the job information is communicated to another clinician. The rejected job information could be represented using different set of icons.

In an embodiment, all job information relating a patient is grouped and displayed to the clinician. For example, if there is any update on the job information relating to a patient, all the job information relating to a particular patient are grouped together and displayed and it could be visually alerted in the work list.

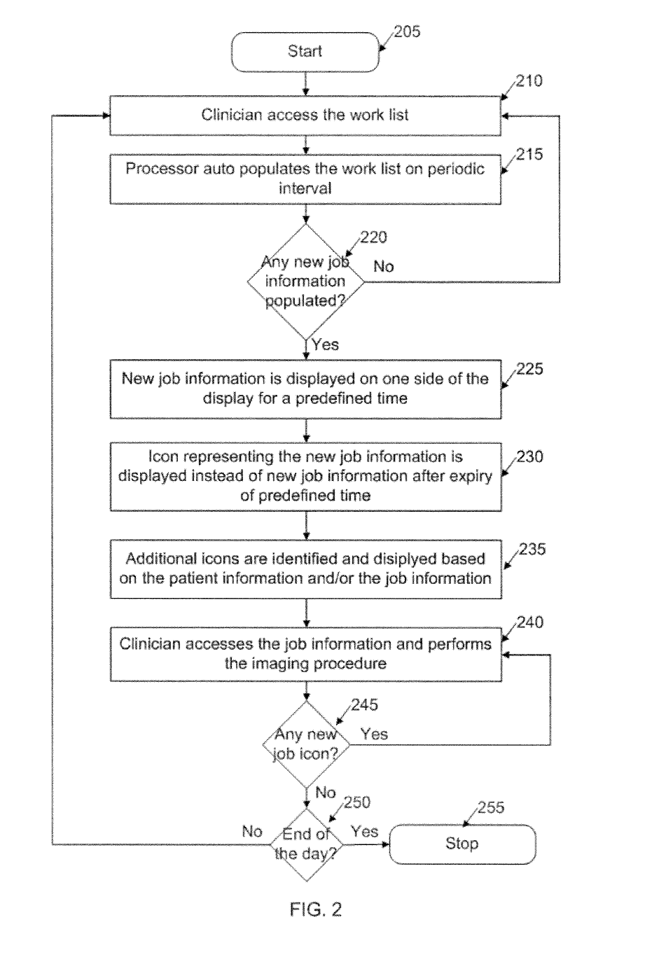

FIG. 2 is a flowchart illustrating detailed workflow for handling an imaging procedure as described in various embodiments of the invention. At step 205, the process is started and the clinician logs in to a system where the schedule of imaging procedures will be displayed to the clinician. At step 210, the clinician accesses the work list. The clinician may be accessing a graphical user interface, which displays the work list. The work list will be displayed to the clinician on a visual display. At step 215, the work list is automatically populated with new job information. The new job information will be accessed from an external data source. The processor associated with the system, will check the data sources at regular interval and will auto populate the job information in the work list. At step 220 a check is made to confirm whether any new job information is populated to the work list. If not, the clinician will wait for the new job information. If the work list is populated with new job information, the new job information will be displayed on the display along with the work list, as at step 225. The new job information along with the patient details could be displayed on one side or corner of the display. The new job information along with the patient information is displayed for a predefined period. At step 230 the new job information is represented using an image icon. The image icon is displayed after expiry of the predefined period and the image icon will indicate that the work list has a new job information. The icon will be displayed until the clinician completes all the job information in the work list.

At step 235, additional icons may be selected based on the nature of the patient information and the new job information. The icons could be predefined and could be available in the system and may be selected automatically while the new job information is being displayed on the display. The job information is represented in detail using additional icons selected based on the patient information and nature of the job information. At step 240, the clinician accesses the new job information and performs the imaging procedure. The new job information can be accessed by clicking on the image icon or by accessing the work list. At step 245 a check is performed by clinician for any new job information. This could be done by accessing the work list or by noticing the image icon representing the new job information. If there is any image icon, the clinician continues the process and accesses the job information and performs the procedure as at step 240. If there is no new job information, a check is performed to ensure whether the scheduled time is over, as at step 250. The clinician might be performing the imaging procedures for a scheduled period in a day and if the schedule period is over, the clinician ends the process as at step 255 and if not, the clinician will access the work flow and continue the process.

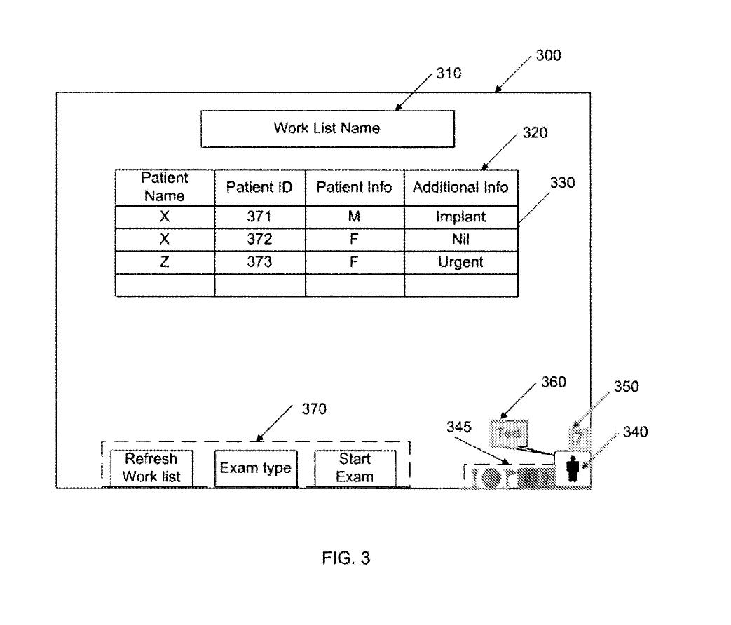

FIG. 3 illustrates a work list displayed with an image icon as described in various embodiments of the invention. The work list 300 is used to maintain the imaging procedures in a hospital. The work list 300 is identified with a work list name 310 indicating the name of the procedure such as mammography exam or X-ray exam. The work list 300 includes plurality of tabs 320 indicating the new job information 330. The job information could include patient name, patient identification details, patient information such as age, gender etc and additional clinical information, which are relevant for the imaging procedure. Plurality of new job information 330 can be populated to the work list 300. In an embodiment, the presence of new job information 330 in the work list 300 is indicated using an image icon 340. The icon 340 represents the new job information 330 in the work list 300. The icon 340 is displayed until the clinician opens the corresponding job information 330 in the work list 300 and performs the imaging procedure.

In an embodiment additional icons 345 are provided to represent patient information or different stages of imaging procedure. The icon 340 can be selected based on the nature of the patient information and action on the job information. Additional icons can represent the priority job information, additional patient information etc.

In an embodiment, the number of new job information can be provided in a number area 350 near to the image icon 340. This will give the clinician an indication about the new job information 330 in the work list without actually opening or looking at the work list or at the new job information 330 listed in the work list 300.

In an embodiment, a comment bar 360 could be displayed along with the image icon 340. This could be used to communicate any additional information to the clinician. The comment bar 360 could include any information about the work list 300 that the receptionist would like to communicate to the clinician or any comments generated by the system based on the job information 330 in the work list 300. For example, comment bar could display “five priority items in the work list” or “Server will be down for five minutes”.

The work list 300 could include additional tabs 370 to start the imaging procedure or to maintain the work list. The additional tabs 370 could include a “refresh list” tab, which will allow the clinician to manually obtain the job information from data sources and update the same in the work list 300.

The layout and arrangement of icons need not be limited to the illustration in the figure. It is to be noted that instead of different icons, different sounds could be provided to indicate different alerts.

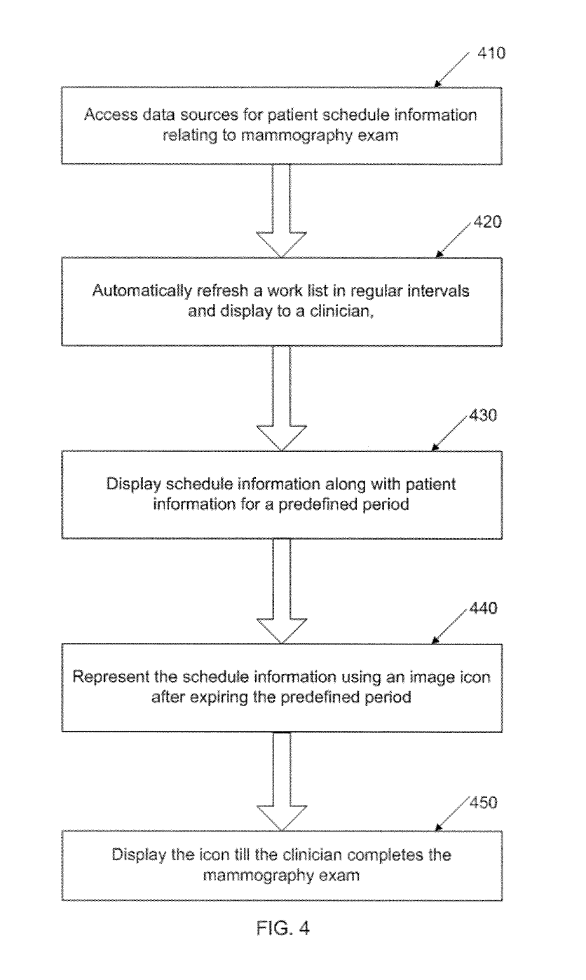

FIG. 4 is a flowchart illustrating method of managing mammography work list as described in an embodiment of the invention. At step 410, a processor that is part of the imaging system or display, accesses at least one data source such as HIS or RIS to get schedule information. The schedule information includes details of the patients scheduled for mammography exam along with patient information. At step 420, the work list is automatically populated with the schedule information. The work list is displayed to the clinician. At step 430, the schedule information along with patient information is displayed to the clinician along with the workflow. This information is displayed for a predefined period, possibly for a very short period. At step 440, the schedule information is represented on the screen using an icon. This icon will indicate that the work list has a new job information. Additional icons can be displayed, which can be selected based on the patient information and/or the nature of the schedule information. At step 450, the icon representing new job information is displayed until the schedule information is accessed and completed by the clinician. Additional icons could be used to represent different stages of the mammography exam.

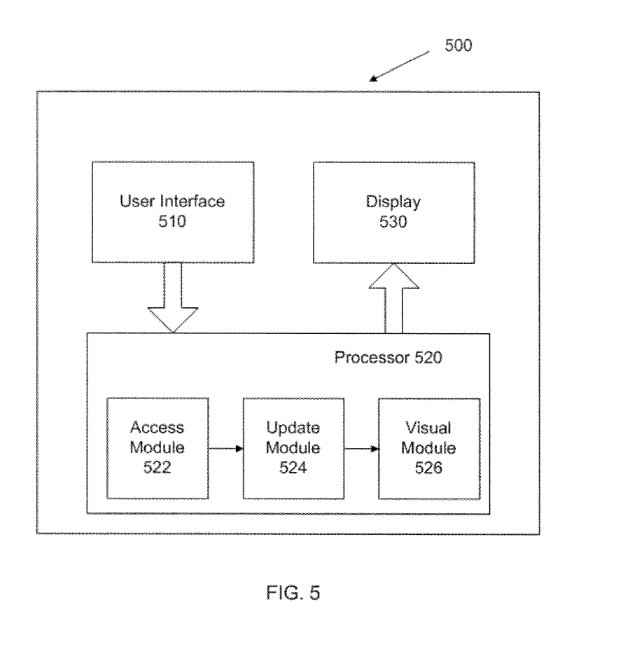

FIG. 5 shows a work list management system 500 as described in an embodiment of the invention. The work list management system 500 maintains a work list that indicates the new job information of schedule information for an imaging procedure and the work list is displayed to the clinician who is performing the imaging procedure. The work list management system 500 includes a user interface 510, a processor 520 and a display 530. The user interface 510 could be a graphical use interface capable of allowing the clinician to interact with the system 500. The work list management system 500 could be a stand-alone system or could be part of an imaging system that performs the imaging procedure. The clinician interacts with the imaging system or the work list management system 500 through the user interface 510.

The processor 520 includes an access module 522, an update module 524 and a visual module 526. The processor 520 is configured to automatically update the work list with new job information. The access module 522 is configured to query any data source at a predefined interval to obtain the new job information. In an embodiment, the access module 522 is configured to access HIS or RIS to obtain scheduled patient information. The update module 524 is configured to update the work list with the information obtained by the access module 522. The update module 524 automatically populates the work list with the new job information, in real time. The visual module 526 is configured to display and alert the clinician about the new job information auto populated into the work list. The visual module 526 is configured to display the new job information on the display 530 for a predefined period. After the expiry of the predefined period, the visual module 526 displays an image icon representing the new job information. The image icon will be displayed until the clinician accesses all the job information in the work list and completes the same. The visual module 522 is further configured to access the new job information, which includes the patient information and any additional information relating to the patient or the medical procedures and represent the new job information in detail using additional image icons. The additional image icons may be selected based on the patient information and/or the nature of the job information. The additional image icons may also be used to represent different stages of the imaging procedure or actions taken on the new job information.

The display 530 is configured to display the work list along with the image icons. The display 530 may include the computer monitor or any other display associated with the processor 520. In case of a graphical user interface 510, the interface 510 may be displayed in the display 530. However there could be different external user interfaces such as joystick, mouse, keypad etc available to assist the user in navigating using the graphical user interface 510.

Embodiments of the present invention can comprise software or firmware instructing a computer to perform certain actions. Some embodiments of the present invention comprise stand-alone workstation computers that include memory, a display, and a processor along with the imaging system. Whether a stand-alone workstation or an imaging system is used, software and/or firmware (hereinafter referred to generically as “software”) can be used to instruct the computer to perform the inventive combination of actions described herein. Portions of the software may have specific functions, and these portions are herein referred to as “modules”. However, in some embodiments, these modules may comprise one or more electronic hardware components or special-purpose hardware components that may be configured to perform the same purpose as the software module or to aid in the performance of the software module. Thus, a “module” may also refer to hardware or a combination of hardware and software performing a function.

The processor 520 may include dedicated hardware, software and/or firmware for performing information processing, or a combination of dedicated hardware and software, or software in combination with a general purpose processor, or a digital signal processor. Once the requirements for such software and/or hardware and/or dedicated hardware are gained from an understanding of the descriptions of embodiments of the invention contained herein, the choice of any particular implementation may be left to a hardware engineer and/or software engineer. However, any dedicated and/or special purpose hardware or special purpose processor is considered subsumed in the block labeled processor 520. The processor may include memory (not shown) for storing the image icons and the memory may include, for example, random access memory (RAM), flash memory, or read-only memory. For purposes of simplicity, devices that can read and/or write media on which computer programs are recorded are also included within the scope of the term “memory.”

Thus various embodiments disclose work list management methods and systems. Thus the method and system has the technical advantage of auto populating the work list at regular intervals and the clinician is alerted about a new job information in the work list.

The advantages of the method and system described includes ability of the system to automatically access the newly scheduled patients and alert the clinician automatically about the same. The system automatically queries the data sources to obtain the schedule information. The image icon will be shown on the display and it will alert the clinician about new/un-assisted patients.

Further, since the work list updating is automated, the clinician need not have to refresh the work list manually to get the schedule information. This saves lots of time and the clinician will always be alerted as the schedule information is shown as an image icon in the display, which will be visible to the clinician even while doing the imaging procedure.

As used herein, an element or step recited in the singular and proceeded with the word “a” or “an” should be understood as not excluding plural elements or steps, unless such exclusion is explicitly stated. Furthermore, references to “one embodiment” of the present invention are not intended to be interpreted as excluding the existence of additional embodiments that also incorporate the recited features. Moreover, unless explicitly stated to the contrary, embodiments “comprising” or “having” an element or a plurality of elements having a particular property may include additional such elements not having that property. Moreover, the terms “computer” and “processor” are used interchangeably herein to refer to either specialized hardware to perform digital signal processing, control, data manipulation, and/or calculations, or a general purpose computer that can be programmed to perform the same functions and/or adapted to interface with external digital signals.

Exemplary embodiments are described above in detail. The assemblies and methods are not limited to the specific embodiments described herein, but rather, components of each assembly and/or method may be utilized independently and separately from other components described herein. Further the steps involved in the workflow need not follow the sequence in which there are illustrated in figures and all the steps in the work flow need not be performed necessarily to complete the method.

While the invention has been described with reference to preferred embodiments, those skilled in the art will appreciate that certain substitutions, alterations and omissions may be made to the embodiments without departing from the spirit of the invention. Accordingly, the foregoing description is meant to be exemplary only, and should not limit the scope of the invention as set forth in the following claims.

What is claimed is:

1. A method of maintaining a work list for medical imaging procedures comprising:
accessing at least one data source by a processor for job information relating to medical imaging procedures;
automatically refreshing a work list in regular intervals; the work list being displayed to a clinician performing the imaging procedure; and
generating at least one alert based on the new job information in the work list.

2. The method as claimed in claim 1, wherein accessing at least one data source further comprises querying a job information repository on regular intervals for new job information.

3. The method as claimed in claim 1, wherein refreshing the work list comprises automatically populating the work list with new job information.

4. The method as claimed in claim 1, wherein generating at least one alert based on the new job information comprises alerting the clinician about the new job information by visual or audio alert and maintaining the alert until an action is taken on the new job information.

5. The method as claimed in claim 1, wherein generating at least one alert based on the new job information comprises:
displaying new job information along with patient information for a predefined period;
representing the new job information using an image icon after expiry of the predefined period.

6. The method as claimed in claim 5, wherein representing the new job information using the image icon comprises representing the new job information as an image icon, until the operator accesses all new job information in the work list.

7. The method as claimed in claim 6, further comprising representing actions taken on the new job information and details of the new job information by additional image icons.

8. The method as claimed in claim 1, further comprising indicating the number of new job information in the work list adjacent to the image icon.

9. The method as claimed in claim 1, further comprising grouping all new job information relating to patient and displaying to the clinician while alerting the clinician about new job information.

10. The method as claimed in claim 1, wherein the step of alerting further comprises generating different types of alerts based on patient's clinical and non clinical information and representing the alerts using additional icons.

11. A method of managing a mammography work list comprising:
accessing data sources for schedule information relating to mammography exam;
automatically refreshing a work list in a regular interval; the work list being displayed to a clinician performing the exam;
displaying the schedule information along with patient information for a predefined period; and
representing the schedule information using an image icon after expiring the predefined period.

12. The method as claimed in claim 11, further comprising displaying the image icon until the clinician completes all the scheduled mammography exams.

13. A work list management system for medical imaging operation comprises:
an interface for a clinician to login and access work list;
a processor comprising:
an access module configured to access at least one job storage device;
an update module for updating the work list in substantially real time;
a visual module configured to alert the clinician about an new job information in the work list; and
a display for displaying the work list.

14. The system as claimed in claim 13, wherein the visual module is configured to display the job information for a predefined period while updating the work list, and displaying as image icon after expiry of the predefined interval.

15. The system as claimed in claim 13, wherein the visual interface module is configured to select and display additional icons based on nature of patient information and modify the additional icons based on actions pending on the job information.
